(19) United States
(12) Patent Application Publication
(10) Pub. No.: US 2010/0042219 A1
(43) Pub. Date: Feb. 18, 2010

(54) EXPANDABLE CAGE FOR INTERVERTEBRAL BODY FUSION

(76) Inventors: Mark Darryl Antonacci, Fort Lee, NJ (US); Alexis Paul Shelokov, Dallas, TX (US)

Correspondence Address: STORM LLP, BANK OF AMERICA PLAZA, 901 MAIN STREET, SUITE 7100, DALLAS, TX, 75202, US

(21) Appl. No.: 12/191,781

(22) Filed: Aug. 14, 2008

(51) Int. Cl. A61F 2/44; A61M 29/00

(52) U.S. Cl. 623/17.16; 623/17.11; 606/191

(57) ABSTRACT

An expandable cage for enhancing fusion of adjacent vertebral bodies includes a housing having a top and a bottom surface. A flexible mesh couples the top and bottom surfaces to allow movement of the top and bottom plate members relative to one another. An aperture formed on the expandable cage receives a selected material. The flexible mesh is in a normally collapsed position but enables the top and bottom plate members to move farther relative to one another when the housing is injected with the selected material. A portion of the flexible mesh expands laterally out of the housing when the housing is injected with the selected material. The top and bottom surfaces are made from carbon fiber, titanium, steel, implantable plastics, or other suitable material. The injected material may be a bone graft or a bone graft substitute (e.g., bone morphogenetic protein).

### TECHNICAL FIELD

This invention relates to surgical instruments and, more particularly, to an expandable cage for intervertebral body fusion.

### BACKGROUND OF THE INVENTION

A ruptured or damaged disk may cause severe pain in the back or neck. The damaged disk is often surgically removed and replaced with an implant such as a cage. The cage is inserted into the cavity between two adjacent vertebral bodies. The cage helps stabilize adjacent vertebral bodies and promotes fusion between the vertebral bodies.

Various type of cages have been developed for use as implants and for promoting fusion between adjacent vertebral bodies. The cages are sometimes filled with bone grafts and bone graft substitutes such as autografts and allografts. Improvement in the design and construction of cages is desired so that the cage may efficiently promote bone fusion and healing.

### SUMMARY OF THE DISCLOSURE

In one embodiment, an expandable cage for enhancing fusion of adjacent vertebral bodies includes a housing having a top and a bottom plate member. A flexible mesh is attached to the top and bottom plate members to create the enclosed housing. The flexible mesh allows movement of the top and bottom plate members relative to one another. An aperture is formed on the expandable cage to receive one or more selected materials. The flexible mesh is normally in a collapsed position but allows the top and bottom plate members to move relative to one another when the housing is injected with the selected materials. A portion of the flexible mesh expands laterally out of the housing when the housing is injected with the selected materials.

The top and bottom plate members are made from carbon fiber, titanium, steel, implantable plastic, or other suitable material. The selected materials may include allograft, autograft, bone morphogenetic protein (BMP), bone marrow, stem cells, and other materials.

In another embodiment, an expandable cage for an interbody implant for enhancing fusion of adjacent vertebral bodies includes a housing formed by a top and a bottom plate and a side wall having at least one opening. The opening is covered by a flexible mesh bag in a normally folded state but expanding out of the opening when the cage is injected with selected materials. The flexible mesh bag includes holes enabling at least one of the selected material to flow out.

### BRIEF DESCRIPTION OF THE DRAWINGS

FIG. 1A illustrates an expandable cage in accordance with one embodiment.

FIG. 1B illustrates the cage after being injected with a selected material.

FIG. 2 illustrates an embodiment in which a flexible mesh bag expands only vertically.

FIG. 3A shows the cage being inserted between two vertebral bodies.

FIG. 3B shows the cage in a collapsed form being implanted between the vertebral bodies.

FIG. 3C shows the cage in its expanded form following injection of the selected material.

FIG. 4A illustrates an expandable cage in accordance with another embodiment.

FIG. 4B shows lateral expansion of the cage.

### DETAILED DESCRIPTION OF THE EMBODIMENTS

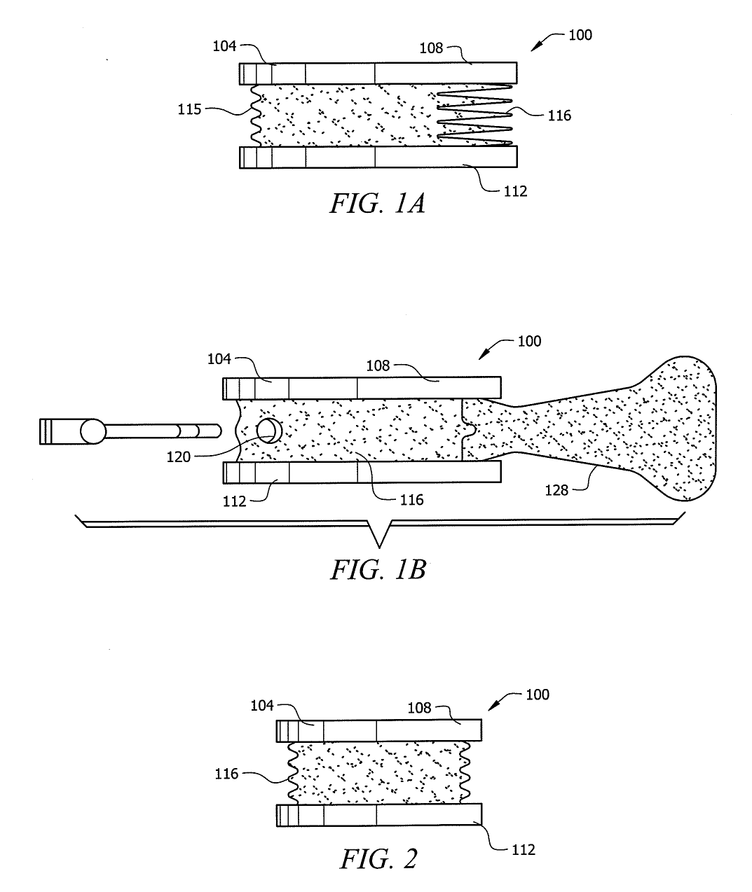

FIG. 1A illustrates an expandable cage 100 in accordance with one embodiment. The cage 100 is configured to maintain a desired gap between adjacent vertebrae to provide stability. Also, the cage 100 enhances fusion of adjacent vertebral bodies.

The cage 100 includes a housing 104 formed by plate members 108 and 112. The plate members 108 and 112 may each be made from carbon fiber, steel, implantable plastic or other suitable material. The plate members 108 and 112 are suitable for implantation in human body.

A flexible mesh 116 forms the side wall of the housing 104. In one embodiment, the flexible mesh 116 is made from a soft material suitable for implantation in human body. The flexible mesh 116 may be made from nylon or other suitable material. In one embodiment, the flexible mesh 116 is made from a biodegradable material that degrades over a period of time. The flexible mesh 116 is attached to the plate members 108 and 112. The flexible mesh 116 may be annealed, press fitted, sewn, bonded or attached in other suitable manner to the plate members 108 and 112.

The flexible mesh 116 is normally in a folded position as shown in FIG. 1A, but allows vertical movement of the plate members 108 and 112. When a selected material is injected into the cage 104, the injected material forces the plate members 108 and 112 to move vertically, thus expanding the cage 104.

FIG. 1B illustrates the cage 100 after being injected with the selected material. In one embodiment, the cage 100 includes an aperture 120 through which the selected material may be injected into the cage 100. An injector 124 shown in FIG. 1B can be utilized to inject the selected material under pressure. Under pressure from the injected material, the plate members 108 and 112 move vertically thus providing vertical expansion of the cage 100. Also, a portion of the flexible mesh 128 expands laterally in a bag-like shape. When fully expanded, the flexible mesh 116 forms the side wall of the cage 100, and a portion of the flexible mesh extends out in a bag-like form 128. Depending on the design, a portion of the flexible mesh may extend out laterally, or may extend out laterally with another portion extending vertically. In some implementations, the flexible mesh 116 may extend out medially, ventrally or dorsally.

In one embodiment, a portion of the flexible mesh 116 is designed to extend out and fill in space between the adjacent vertebral bodies. The flexible mesh 116 is constructed to allow certain selected material to flow out of the mesh to enhance fusion of the adjacent vertebral bodies. In one embodiment, the flexible mesh 116 has holes that allow certain selected material to flow out to enhance fusion of the intervertebral bodies. For example, the flexible mesh 116 may have holes of suitable size to allow the release of certain selected material but may retain other materials. For example, the cage 100 may be injected with several material including a bone graft material such as allograft or autograft and bone morphogenetic protein (BMP). The flexible mesh 116 may be constructed to allow the bone morphogenetic protein (BMP) to flow out of the mesh 116 but retain the allograft or autograft. Thus, the allograft or autograft provides the structural stability by maintaining the gap between the plate members 108 and 112, while the BMP induces bone fusion and healing of adjacent vertebral bodies. In one embodiment, the flexible mesh 116 may have holes of suitable size to retain certain selected materials (e.g., allograft or autograft) but allow certain selected materials (e.g., BMP, stem cells, bone marrow cells, etc.) to flow out of the mesh 116 to the adjacent region in order to promote fusion and healing.

In one embodiment, the cage 100 may be injected with solid particulates of bone graft or bone graft substitute materials such as allograft or autograft that are retained inside the flexible mesh 116 to provide structural stability and maintain the required gap between adjacent vertebrae. Thus, the mesh 116 does not allow these solid particulates (e.g., autograft, allograft or other bone substitutes) to flow out of the mesh 116.

The cage 100 may also be injected with certain selected materials (e.g., BMP, stem cells, bone marrow cells), which flow out of the flexible mesh 116. These selected materials may be in liquid, slurry, powder or other suitable form allowing them to flow out of the flexible mesh 116 over a period of time to induce and promote healing and fusion. The holes in the flexible mesh 116 may be sized suitably to retain the solid particulates but allow the BMP, stem cells, bone marrow cells to flow out of the mesh 116. For example, the flexible mesh 116 may have holes that allow microscopic particles (e.g., BMP, stem cells, bone marrow cells) to flow out of the mesh 116 over a period of time. It will be understood by those skilled in the art that the bone graft or bone graft substitute materials may be in crystalline, granular or other forms suitable for retention inside the flexible mesh 116.

The flexible mesh 116 may be made from a soft fiber-type material suitable for implantation in human body. The flexible mesh 116 may, for example, be made from a carbon fiber-type or polyethylene material.

FIG. 2 illustrates and embodiment 200 in which the flexible mesh 116 expands only vertically. As discussed before, the flexible mesh 116 may extend in various directions depending on the design.

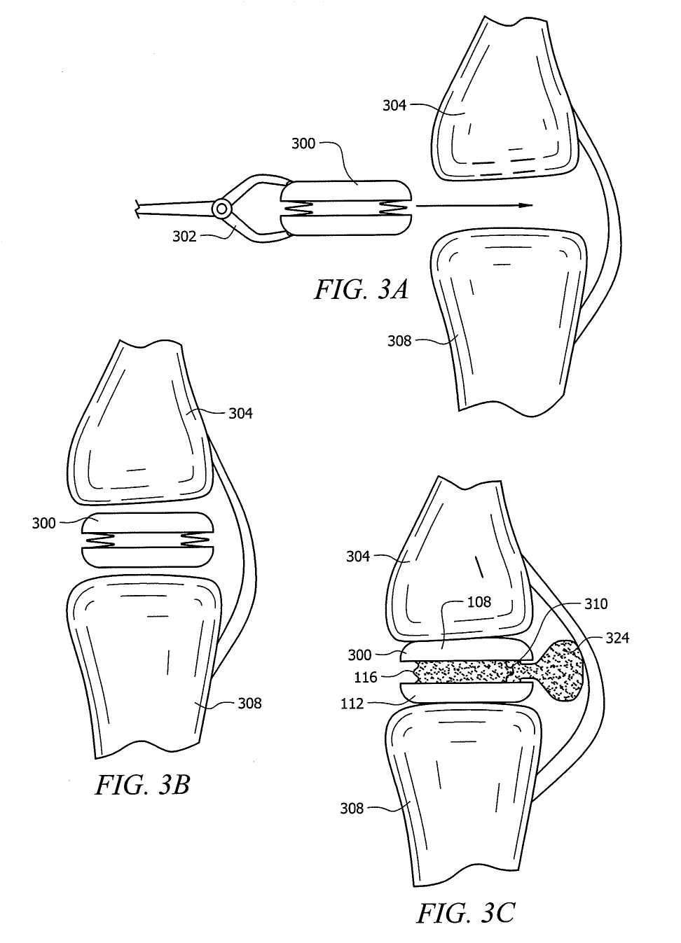

FIG. 3A shows the cage 300, which is normally in a collapsed form, being inserted between two vertebral bodies, 304 and 308, using a surgical instrument 302. FIG. 3B shows the cage 300 in a collapsed form being implanted between the vertebral bodies 304 and 308.

FIG. 3C shows the cage 300 in its expanded form following injection of the selected material. The selected material is preferably injected under pressure. Due to the injection of the selected material, the top and bottom plates 108 and 112 are driven apart, thus expanding the mesh 116. In one implementation, the mesh 116 expands vertically as well as laterally creating a mesh bag 324, which fills up the cavity between the vertebral bodies 304 and 308. In one embodiment, a small valve 310 inside the cage 300 can be utilized to enable the vertical expansion of the mesh 116 and then the lateral expansion to form the mesh bag 324. Thus, the valve 310 enables the mesh 116 to expand initially vertically, and subsequently enables the mesh 116 to expand laterally.

In one embodiment, the top and bottom plates are made from a bio-degradable materials or implantable plastics (e.g., PEEK) suitable for human implantation.

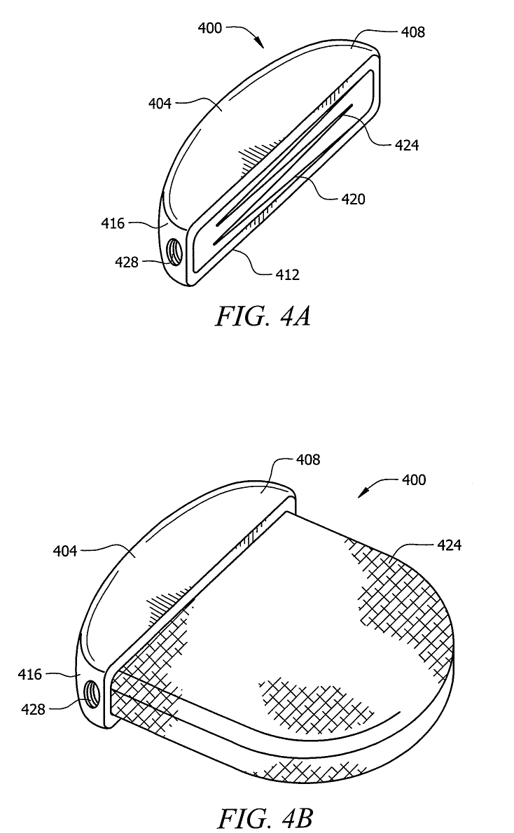

FIG. 4A illustrates an expandable cage 400 in accordance with another embodiment. The expandable cage 400 includes a housing 404 formed by a top plate 408, a bottom plate 412, and a side wall 416. In one embodiment, the housing 404 is bounded on three sides by the side wall 416, but has an opening 420 on one side.

It will be appreciated that in alternative embodiments, the cage 400 may have a plurality of openings 420 on one or more sides. Thus, a mesh bag may expand out of each of the openings to fill or expand any disk space (i.e., space between two adjacent vertebra). Depending on the design, the mesh bags may expand out of the cage 400 in any direction (dorsal, ventral, lateral, medial or combination thereof) to fill the disk space. Also, in one embodiment the cage 400 may have a circular shape having a fixed height that may deploy a mesh bag like a doughnut in every direction. Thus, in one embodiment, the top and bottom plates may have circular shape, and the mesh bag may expand out in every direction like a doughnut. The mesh bag may have a volumetric design.

The opening 420 is covered by a flexible mesh bag 424 having an area greater than the opening 420. In one embodiment, the opening 420 is sealed by the flexible mesh 424 having dimensions greater than the dimension of the openings 420, thus enabling the flexible mesh 424 to be in a folded state until inflated by one or more selected materials injected into the cage 400.

The top plate 408, the bottom plate 412 and the side wall 416 may be formed by carbon fiber, steel, titanium, implantable plastic or any other suitable material. In one embodiment, the top plate 408, the bottom plate 412 and the side wall 416 are formed by a bio-degradable plastic such as PEEK or other similar material suitable for implantation in human body. As shown in FIG. 4A, the housing 404 has a fixed height, length and width to fit between adjacent vertebral bodies following surgical removal of a disk. It will be understood that the housing 404 may be constructed of various forms, shapes and sizes. Also, the housing 404 may have two or more openings covered (or sealed) by a flexible mesh.

One or more selected materials such as bone graft, bone graft substitutes (e.g., allograft, autograft), bone morphogenetic protein (BMP), stem cells, bone marrow, or other material is injected into the cage through an aperture or hole 428. As discussed before, the bone graft or bone graft substitutes may be in particulate form or may formed as pellets or other suitable form. The bone graft or bone graft substitute is injected to fill in the cage, while the BMP, stem cells, bone marrow is injected to induce bone fusion, growth and healing. In one embodiment, the bone graft of bone graft substitutes are retained inside the mesh bag 424, but the stem cell, BMP and other selected materials flow out of the mesh bag 424 through tiny holes on the mesh bag 424 to induce bone fusion and healing. Over a period of time, the bone graft or bone graft substitutes may solidify inside the mesh bag 424 to provide structural support to the adjacent vertebrae, while the BMP, stem cell, bone marrow or other selected material may flow out of the mesh bag 424 to promote bone fusion and healing.

The mesh bag 424 is normally in a folded state, but is driven out by the injected material, thereby providing lateral expansion of the cage 400 as shown in FIG. 4B. It will be appreciated, depending on the design, a portion of the mesh bag 424 may extend vertically, medially, dorsally or ventrally as well. Also, the expandable cage 400 may have different forms or shapes including lordotic, parallel or oblique.

In one implementation, after removal of a disk, one of the embodiments of the cage shown in FIGS. 1-4 is inserted using an insertion jig which holds the cage in a collapsed position between two adjacent vertebral bodies. For example, the cage may be inserted from the back to a lateral position in the intervertebral cavity following removal of the disk and then gradually inflated, preferentially expanding the top and the bottom plates to stabilize the space and then expanding the bag.

In another embodiment, the cage is inserted following diskectomy using an insertion jig which holds the cage between two adjacent vertebral bodies, with the mesh bag in a collapsed position. The mesh bag is then gradually inflated by injecting one or more selected materials, preferentially expanding out of the cage into the intervertebral cavity, filling the intervertebral cavity.

In another embodiment, the cage is bounded by top and bottom plates and one or more side walls. The side walls may have one or more openings, each covered or sealed by a flexible mesh bag. Thus, in one embodiment, the cage may have an opening on a first side wall and another opening on a second side wall. The cage is injected with one or more selected materials, causing one or more flexible mesh bags to expand out of the cage.

The foregoing description of illustrated embodiments is not intended to be exhaustive or to limit the disclosure to the precise forms disclosed herein. While specific embodiments and examples are described herein for illustrative purposes only, various equivalent modifications are possible within the spirit and scope of the disclosure, as those skilled in the relevant art will recognize and appreciate. As indicated, these modifications may be made in light of the foregoing description of illustrated embodiments and are to be included within the spirit and scope of the disclosure.

Thus, while the disclosure has been described herein with reference to particular embodiments thereof, a latitude of modification, various changes and substitutions are intended in the foregoing disclosures, and it will be appreciated that in some instances some features of embodiments will be employed without a corresponding use of other features without departing from the scope and spirit of the disclosure as set forth. Therefore, many modifications may be made to adapt a particular situation or material to the essential scope and spirit of the disclosure. It is intended that the disclosure not be limited to the particular terms used in following claims and/or to the particular embodiment disclosed, but that the disclosure will include any and all embodiments and equivalents falling within the scope of the appended claims. Thus, the scope of the invention is to be determined solely by the appended claims.

What is claimed is:

1. An expandable cage for enhancing fusion of adjacent vertebral bodies, comprising:
a housing having a top and a bottom plate member;
a flexible mesh coupling the top and bottom plate members to allow movement of the top and bottom plate members relative to one another;
an aperture formed on the expandable cage to receive a selected material;
the flexible mesh being in a normally collapsed position but enabling the top and bottom plate members to move relative to one another when the housing is injected with the selected material, a portion of the flexible mesh expanding laterally out of the housing when the housing is injected with the selected material.

2. The expandable cage of claim 1, wherein the top and bottom plate members are made from carbon fiber.

3. The expandable cage of claim 1, wherein the top and bottom plate members are made from titanium or steel.

4. The expandable cage of claim 1, wherein the top and bottom plate members are made from an implantable material.

5. The expandable cage of claim 1, wherein the selected material is a bone graft material.

6. The expandable cage of claim 1, wherein the selected material is a bone graft substitute.

7. The expandable cage of claim 1, wherein the selected material is a bone morphogenetic protein (BMP).

8. The expandable cage of claim 1, wherein the housing is dimensioned to fit between adjacent vertebral bodies following removal of a vertebral disk.

9. The expandable cage of claim 1, wherein the injected material flows out of the flexible mesh to induce bone fusion.

10. The expandable cage of claim 1, further comprising an interior valve enabling a vertical expansion of the cage prior to a lateral expansion of the flexible mesh.

11. The expandable cage of claim 1, further comprising an interior valve enabling a preferential vertical expansion of the cage prior to a lateral expansion of the flexible mesh.

12. An expandable cage for enhancing fusion of adjacent vertebral bodies, comprising:
a housing having a top and a bottom plate members coupled by a flexible mesh;
the flexible mesh normally being in a collapsed state with the flexible mesh being folded, the housing expanding vertically when injected with a selected material, a portion of the flexible mesh expanding out of the housing to form a mesh bag filled with the selected material, the flexible mesh having holes enabling the selected material to flow out to enhance fusion.

13. The expandable cage of claim 12, wherein the top and bottom plate members are made from carbon fiber.

14. The expandable cage of claim 12, wherein the top and bottom plate members are made from titanium.

15. The expandable cage of claim 12, wherein the selected material is a bone graft material.

16. The expandable cage of claim 12, wherein the selected material is a bone graft substitute.

17. The expandable cage of claim 12, wherein the selected material is a bone morphogenetic protein (BMP).

18. The expandable cage of claim 12, wherein the housing is dimensioned to fit between adjacent vertebral bodies following removal of a vertebral disk.

19. An expandable cage for enhancing fusion of adjacent vertebral bodies, comprising a housing formed by a top and a bottom plate member and at least one side wall having an opening, the opening being covered by a flexible mesh bag having a surface area greater than the opening, the flexible mesh bag in a normally folded state but expanding out of the opening when the cage is injected with a selected material, the flexible mesh bag having holes enabling the selected material to flow out of the flexible mesh bag.

20. The expandable cage of claim 19, wherein the top and bottom plate members are made from carbon fiber.

21. The expandable cage of claim 19, wherein the top and bottom plate members are made from titanium.

22. The expandable cage of claim 19, wherein the selected material is a bone graft material.

23. The expandable cage of claim 19, wherein the selected material is a bone graft substitute.

24. The expandable cage of claim 19, wherein the selected material is a bone morphogenetic protein (BMP).

25. The expandable cage of claim 19, wherein the housing has a fixed height.

26. An expandable cage for an interbody implant for enhancing fusion of adjacent vertebral bodies, comprising a housing formed by a top and a bottom plate and a side wall having at least one opening, the opening being covered by a flexible mesh bag in a normally folded state but expanding out of the opening when the cage is injected with a selected material, the flexible mesh bag having holes enabling the selected material to flow out, the housing dimensioned to fit between the intervertebral bodies.

27. The expandable cage of claim 26, wherein the top and bottom plates are made from carbon fiber.

28. The expandable cage of claim 26, wherein the top and bottom plates are made from an implantable material.

29. The expandable cage of claim 26, wherein the selected material is a bone morphogenetic protein (BMP).

30. An expandable cage for an interbody implant for enhancing fusion of adjacent vertebral bodies, comprising a housing formed by a top and a bottom plate, the housing being bounded on the side by at least one side wall and having an opening, the opening being covered by a flexible mesh bag in a normally folded state, the flexible mesh bag expanding out of the opening when the cage is injected with selected materials, the flexible mesh bag having holes enabling one or more selected materials to flow out, the housing sized to fit between the intervertebral bodies.
